(19) United States
(12) Patent Application Publication
(10) Pub. No.: US 2025/0229029 A1
(43) Pub. Date: Jul. 17, 2025

(54) MEDICAL SYSTEMS, DEVICES, AND RELATED METHODS FOR DELIVERY OF TREATMENT AGENT(S)

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); Kevin L. BAGLEY, Natick, MA (US)

(73) Assignees: Boston Scientific Scimed, Inc., Maple Grove, MN (US); Boston Scientific Medical Device Limited, Galway (IE)

(21) Appl. No.: 19/016,476

(22) Filed: Jan. 10, 2025

Related U.S. Application Data
(60) Provisional application No. 63/620,069, filed on Jan. 11, 2024.

(51) Int. Cl. A61M 5/31; A61J 1/20

(52) U.S. Cl. CPC A61M 5/31; A61J 1/20

(57) ABSTRACT

Medical systems, devices, and related methods for delivery of treatment agent(s) are described. The medical device includes a container. The container includes a chamber and a distal cap. The distal cap of the container includes one or more holes therethrough. The chamber includes a balloon or a pouch, and a fluid cavity. The balloon or pouch radially surrounds the fluid cavity. Transitioning the balloon or pouch from a deflated configuration to an inflated configuration or from the inflated configuration to the deflated configuration urges a fluid within the fluid cavity through one or more holes of the distal cap.

### CROSS-REFERENCE TO RELATED APPLICATIONS

This application claims the benefit of priority to U.S. Provisional Application No. 63/620,069, filed on Jan. 11, 2024, which is incorporated herein by reference in its entirety.

### TECHNICAL FIELD

The disclosure relates generally to systems, devices, and methods for delivering one or more treatment agents. More specifically, aspects of the disclosure pertain to systems, devices, and/or methods for delivering viscous fluid or one or more treatment agents to a target site, via medical devices, such as endoscopes.

### BACKGROUND

Bleeding ulcers or other wound sites, for example, in a subject's gastrointestinal (GI) tract, may be treated via various treatments, including injection therapies. Injection therapies may require the delivery of a fluid, oftentimes, a highly viscous fluid, to the wound site(s) to form a protective layer that helps to minimize delayed bleeds, potential perforations, stricture formations, etc. Devices for fluid delivery may include one or more fluid channels, with the one or more fluid channels each having a small diameter. Highly viscous fluids may require a large amount of force or pressure to flow through narrow fluid channels. Common fluid delivery devices, e.g., syringes, generally deliver fluid when a push force, i.e., a distal force, is applied to a feature of such devices, and a push force may be smaller than desirable. Applying a large amount of force or pressure to urge the viscous fluid distally may be difficult and/or time-consuming for the operator. Additionally, applying the large amount of force or pressure may increase the risk of one or more components of the medical device deforming, breaking, or otherwise failing.

### SUMMARY

The disclosure includes systems, devices, and methods for delivering one or more treatment agents, e.g., methods of delivering fluid to a target site of a subject, for example, to help heal an ulcer and/or to perform hemostasis. Each of the aspects disclosed herein may include one or more of the features described in connection with any of the other disclosed aspects.

For example, the disclosure includes a medical device. The medical device may include a container. The container may include a chamber and a distal cap. The distal cap of the container may include one or more holes therethrough. The chamber may include a balloon or pouch, and a fluid cavity. The balloon or pouch may radially surround the fluid cavity. Transitioning the balloon or pouch from a deflated configuration to an inflated configuration or from the inflated configuration to the deflated configuration may urge a fluid within the fluid cavity through one or more holes of the distal cap. The chamber may include the balloon. The medical device may further include a barrier layer positioned between the balloon and the fluid cavity. The barrier layer may include one or more bellows.

The medical device may include one or more of the following features. The chamber may include a balloon and a barrier layer positioned between the balloon and the fluid cavity. The container may be a first container. The chamber may be a first chamber. The medical device may further include a second container. The second container may include a second chamber. The second chamber may include one or more holes in fluid communication with the fluid cavity of the first chamber. A transition of the balloon from the inflated configuration to the deflated configuration may urge another fluid from the second chamber into the fluid cavity of the first chamber. The fluid cavity may include a fluid. The fluid may be fibrin. The second container may include a second chamber. The second chamber may include the fluid. The first container may include a first outer circumference and a first inner circumference. The balloon, barrier layer, and the fluid cavity may be positioned between the first outer circumference and the first inner circumference. The medical device may be positioned at a distal end of a shaft of an endoscope. The first inner circumference may include a first open space radially inward of the first inner circumference. The first open space may define a first passage sized and shaped to receive the shaft of the endoscope. The second container may include a second outer circumference and a second inner circumference. The second inner circumference may include a second open space radially inward of the second inner circumference. The second open space may define a second passage sized and shaped to receive the shaft of the endoscope. The distal cap of the first container may include a circular electrical strip with one or more light sources thereon. The circular electrical strip may be positioned radially outward of the first inner circumference and radially inward of the first outer circumference. The first container may include a channel positioned on an exterior surface of the first container. The channel may include a lumen. The lumen may include an electrical conduit. The circular electrical strip may include a tab extending radially outward of the electrical strip. The electrical conduit may be electrically connected to the tab of the electrical strip to supply electrical power to the one or more light sources of the electrical strip.

The second container may include a distal cap and a proximal cap. The distal cap may include one or more distal outer holes and one or more distal inner holes. The proximal cap may include one or more proximal holes. The medical device may include one or more tubes configured to provide and/or remove gas or fluid from the balloon. A distal opening of each of the one or more tubes may be in fluid communication with the balloon. The one or more distal outer holes and the one or more proximal holes may each be sized and shaped to receive a portion of one of the one or more tubes. The distal cap of the first container may be positioned near distal ends of the inner circumference and the first outer circumference. The distal cap of the second container may be positioned at proximal ends of the first inner circumference and the first outer circumference to seal the first container. The distal cap of the second container may be positioned at distal ends of the second inner circumference and the second outer circumference. The proximal cap may be positioned at proximal ends of the second inner circumference and the second outer circumference to seal the second container. The proximal end of the first container may be affixed to a distal end of the second container.

The one or more holes of the distal cap may extend, in a proximal direction to a distal direction, radially inward relative to a central longitudinal axis of the medical device.

The disclosure also includes a medical system. The medical system may include a first container. The first container may include a first chamber and a distal cap. The medical system may include a second container. The second container may include a second chamber, a distal cap, and a proximal cap. The medical system may include a syringe and one or more tubes extending from a distal end of the syringe to the first container. The first chamber may include a balloon and a fluid cavity. The balloon may radially surround the fluid cavity. The syringe, the one or more tubes, and the balloon may be in fluid communication. Transitioning the balloon from a deflated configuration to an inflated configuration by delivering a fluid or a gas from the syringe may urge fluid within the fluid cavity through one or more holes of the distal cap.

The medical system may include one or more of the following features. The one or more tubes may extend through the distal cap and the proximal cap of the second container. The one or more tubes may be fluidly isolated from the second chamber. The distal cap of the second container may include one or more holes. The fluid cavity, one or more holes of the distal cap of the second container, and the second chamber may be in fluid communication.

The disclosure further includes a medical system. The medical system may include a first container. The first container may include a first chamber and a distal cap. The medical system may include a second container. The second container may include a second chamber. The medical system may also include a syringe and one or more tubes extending from a distal end of the syringe. The first chamber may include a balloon and a fluid cavity. The balloon may radially surround the fluid cavity. The first chamber may include a barrier layer positioned between the balloon and the fluid cavity. A distal end of the second container may be affixed to a proximal end of the first container. Transitioning the balloon from a deflated configuration to an inflated configuration by manipulating the syringe may urge fluid within the fluid cavity through one or more holes of the distal cap.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a perspective view of an exemplary medical device.

FIG. 2 depicts a transparent view of a distal portion of the medical device, with the distal portion of the medical device coupled to a distal end of an insertion device.

FIG. 3A depicts a perspective view in a distal to proximal direction of a feature of the medical device.

FIG. 3B depicts a perspective view in a proximal to distal direction of the feature of the medical device of FIG. 3A.

FIG. 4A depicts a perspective view in a distal to proximal direction of another feature of the medical device.

FIG. 4B depicts a perspective view in a proximal to distal direction of another feature of the medical device of FIG. 4A.

FIG. 5A depicts a perspective view of a distal portion of the medical device coupled to the distal end of the insertion device, with various portions of the medical device shown as being transparent.

FIG. 5B depicts another perspective view of the same portion of the medical device of FIG. 5A coupled to the distal end of the insertion device, with various portions of the medical device shown as being transparent.

FIG. 6 depicts a perspective view of another exemplary medical device.

FIG. 7 depicts a cross sectional view of the medical device of FIG. 6.

FIG. 8 depicts a cross sectional view of yet another exemplary medical device.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

Aspects of this disclosure seek to help improve and ease a user's ability to deliver a highly viscous fluid via a device fitted over or around an insertion device (e.g., a medical scope) or a device delivered through a working channel of an insertion device. Additionally, various aspects of this disclosure may help the user perform wound treatment and/or hemostasis within the subject, reduce overall procedure time, reduce overall procedure costs, etc. Each of the embodiments of this disclosure is configured to deliver viscous fluid when a push force, i.e., a distal force, is applied to a respective feature. The presence of a push feature for fluid delivery, as opposed to a pull feature for fluid delivery, may allow for an easier application of force and/or a sufficient amount of force to deliver viscous fluid through the various openings and channels of the embodiments of this disclosure. Examples of fluids (e.g., biocompatible viscous fluids) include, but are not limited to, fibrin, fluids including calcium salts, cyanoacrylates, albumin and glutaraldehyde, poly (ethylene glycol) (PEG), polyurethane, etc. Such fluids may be endoscopically delivered adhesives that help to create a protective layer that minimizes delayed bleeds, potential perforations, and stricture formations. Light sources/lights at a distal end of a medical device may help to cure or set fluids (e.g. adhesives) more quickly. Additionally, an insertion portion/scope of an insertion device may include one or more visualization elements (e.g., a light and/or a camera) to help the user visualize a treatment site. The one or more visualization elements may also help the user to visualize the application of the fluids and/or the setting of the fluids on the treatment site.

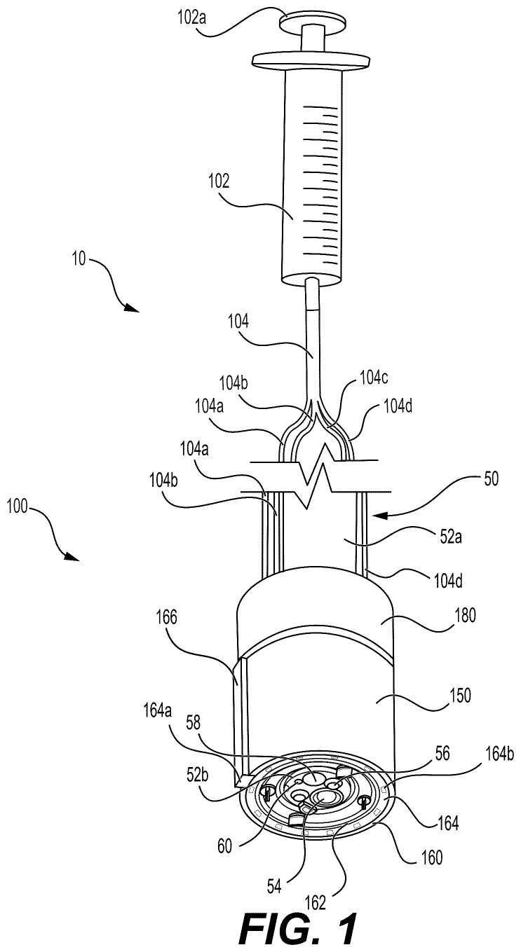

FIGS. 1-5B illustrate a medical device 100. As shown in FIG. 1 one or more portions of medical device 100 may be coupled to another or second medical device. For example, an endoscope 50, to form a medical system 10. Elements of medical system 10 and medical device 100 are not necessarily shown to scale and certain components of medical system 10 may be omitted from the figures for clarity purpose. Medical system 10 may include a syringe 102. Syringe 102 includes a plunger 102a.

Endoscope 50 may include a generally cylindrical tubular shaft 52, and may include a proximal portion (not shown) and a distal portion 52a that terminates at a distal end 52b. Endoscope 50 may include a diameter ranging from 10.5 mm to 12 mm (+/−1 mm), however endoscope 50 may include any diameter. Although not shown, the proximal portion may be include or otherwise be coupled to a handle, for example, including one or more ports, controls, levers, electrical or communication connections, etc. Additionally, endoscope 50 may include one or more internal lumens or working channels (e.g. working channel 54), for example, extending longitudinally through shaft 52. In these aspects, the internal lumens or working channels may extend through the proximal portion and distal portion 52a of shaft 52, for example, terminating distally at one or more distal openings (i.e., in a distal most end of endoscope 50).

Endoscope 50 (i.e., shaft 52 of endoscope 50) is not particularly limited. Shaft 52 may be any size and/or shape configured to be coupled to one or more portions of medical device 100. Additionally, a distal end 52b (e.g. a distal end face) of endoscope 50 may include one or more illumination device(s) or light sources 56 (e.g., one or more LEDs, optical fibers, and/or other illuminators) and/or one or more visualization device(s) or cameras 58 (e.g., one or more camera, one or more image sensors, endoscopic viewing elements, optical assemblies including one or more image sensors and one or more lenses, etc.). Shaft 52 of endoscope 50 may include one or more fluid channels 60. Fluid channel(s) 60 may be a fluid delivery channel and/or may be a suction or negative pressure channel.

Although not shown, one or more portions of endoscope 50 (i.e., a distal portion of shaft 52) may be deflectable, for example, via one or more knobs or other controls on a proximal handle of endoscope 50. In these aspects, shaft 52 of endoscope 50 may be maneuvered while being delivered to the treatment site and/or positioned relative to the treatment site, for example, in a retroflex position, which may be used when the treatment site is in the subject's esophagus, stomach, duodenum, colon, or other portion of the GI tract.

Although the treatment site is discussed as being in the subject's GI tract, this disclosure is not so limited, as the treatment site may be any internal lumen or other tissue within the subject. Potential clinical uses of the embodiments of this disclosure and exemplary viscous fluids include post resection (e.g., endoscopic mucosal resection, endoscopic submucosal dissection, polypectomy), suture line reinforcement, fistula sealing, and post peroral endoscopic myotomy site closure. Additionally, although endoscopes are referenced herein, it will be appreciated that the disclosure encompasses any medical devices having a working channel extending from a proximal end to a distal end, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

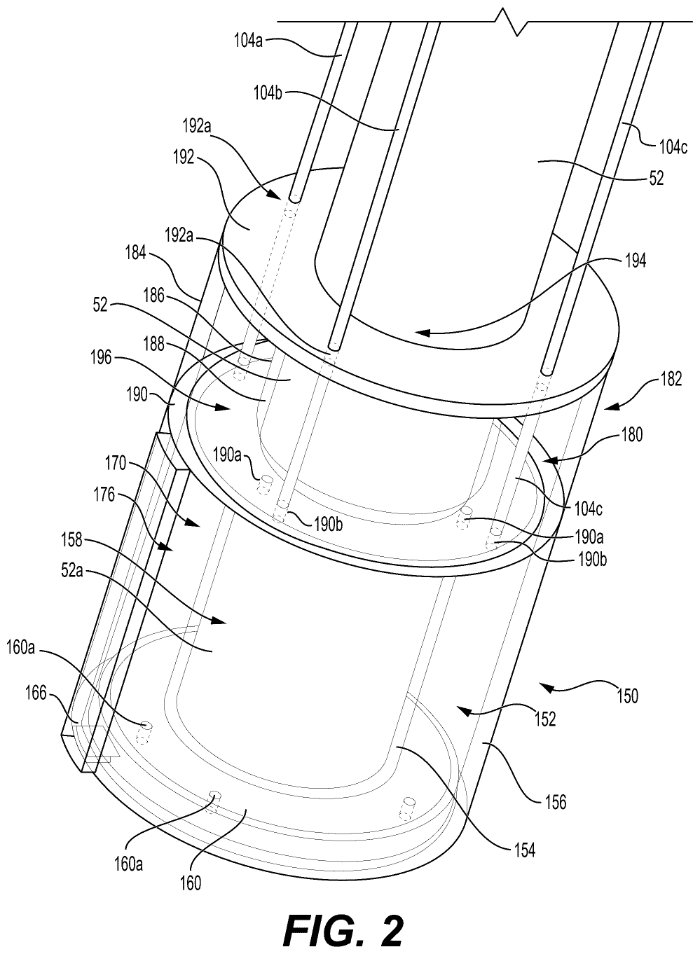

As shown in FIG. 1 and FIG. 2, medical device 100 includes a first (distal) container 150 and a second (proximal) container 180. As discussed below, features of medical device 100 may be coupled to features of a control element (e.g., a syringe 102) via one or more conduits (e.g. one or more tubes 104a, 104b, 104c, and 104d of tubing 104). FIG. 1 illustrates a perspective view of medical system 10 including endoscope 50, medical device 100 coupled/affixed to distal portion 54A of endoscope 50, and syringe 102 coupled to tubing 104 (including tubes 104a, 104b, 104c, and 104d). As discussed in detail below, syringe 102 may be fluidly coupled to medical device 100. FIG. 2 illustrates a perspective view of distal end of endoscope 50 with medical device 100 (including first container 150 and second container 180) coupled/affixed to the distal end of endoscope 50.

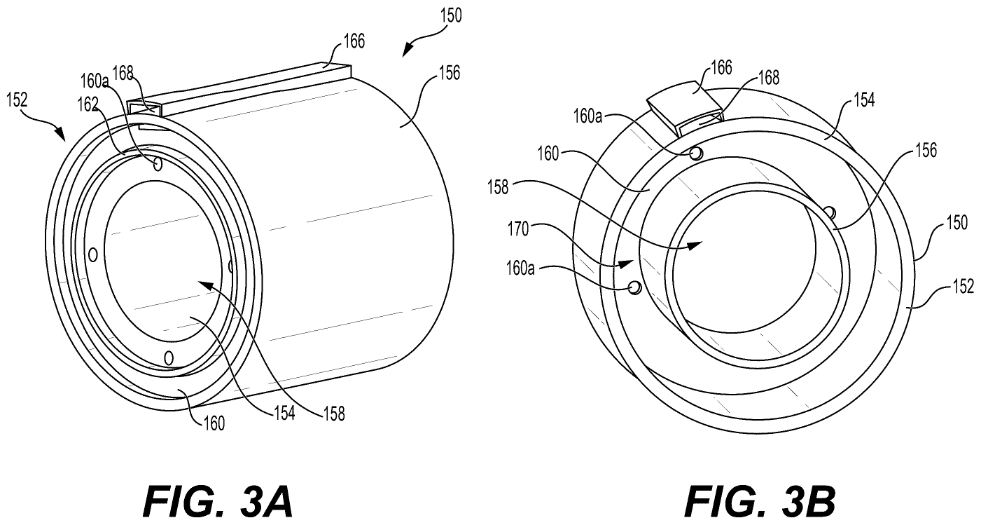

FIGS. 3A and 3B illustrate perspective views of first container 150. For example, FIG. 3A shows a perspective view of a distal end of first container 150 including a distal cap 160 and inner and outer circumferences 154, 156, respectively, and FIG. 3B shows a proximal end of first container 150. As shown in FIGS. 2-3B, 5A-5B, first container 150 may include a casing 152. Casing 152 may be an annular or O-shaped cylindrical structure defining an outer surface or circumference 156 and an inner surface or circumference 154. The open space within inner circumference 154 may define a central opening or passage 158. Passage 158 may be of any suitable shape and/or diameter that may receive or otherwise be positioned around distal portion 52a of shaft 52 of endoscope 50, so that shaft 52 may securely/snugly fit within passage 158. In some embodiments, a surface of casing 152 surrounding passage 158 may be lined with, coated with, or otherwise include one or more frictious surfaces and/or one or more adhesive materials to help secure the coupling between casing 152 and shaft 52 of endoscope 50.

As mentioned, first container 150 may include distal cap 160. Passage 158 may be also extend through distal cap 160, so that distal cap 160 includes a central opening. Distal cap 160 may be O-shaped. Distal cap 160 may be recessed within casing 152 proximally relative to a distal end of outer circumference 156 and may be positioned at a distal end of inner circumference 154. Distal cap 160 may be integrally formed with casing 152 (e.g., fixedly attached to an inner surface of outer circumference 156 and a distal end of inner circumference 154) and may seal a distal end of casing 152. As shown in FIG. 3A, distal cap 160 may include a distally extending protrusion 162. Protrusion 162 may be an annular or O-shaped cylindrical structure. Protrusion 162 may include a greater diameter than inner circumference 154, but a lesser diameter than outer circumference 156. First container 150 may include or otherwise define a chamber 170. Chamber 170 may be defined by a space between the inner circumference 154 and outer circumference 156 proximal to distal cap 160.

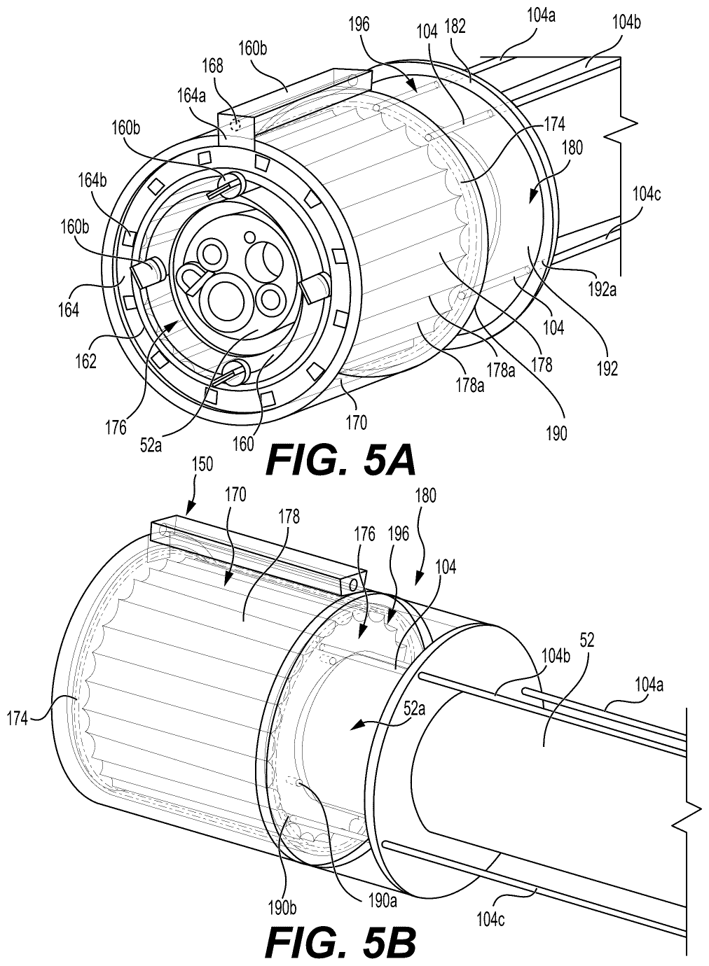

As shown in FIGS. 1 and 5A, medical device 100 may include an electrical strip 164. Electrical strip 164 may include an O-shape. Electrical strip 164 may be sized and shaped to be positioned in the space between protrusion 162 and outer circumference 156. Electrical strip 164 may include one or more light sources 164b (such as an UV and/or VIS-emitting LED(s)). Electrical strip 164 may be configured to transmit electricity and/or signals to the one or more light sources. Electrical strip 164 may include a tab 164a, for example, extending radially outward from electrical strip 164. Distal cap 160 may include a channel 166 extending along an outer surface of distal cap 160. Channel 166 may include a lumen 168. Lumen 168 may be electrically insulated. One or more conductors (not shown) for transmitting and/or conveying electricity (e.g., electrical power) and/or signals may extend through lumen 168. A distal end(s) of the one or more conductors may be electrically and/or communicatively connected to tab 164a and thereby connected to electrical strip 164, and the one or more light sources.

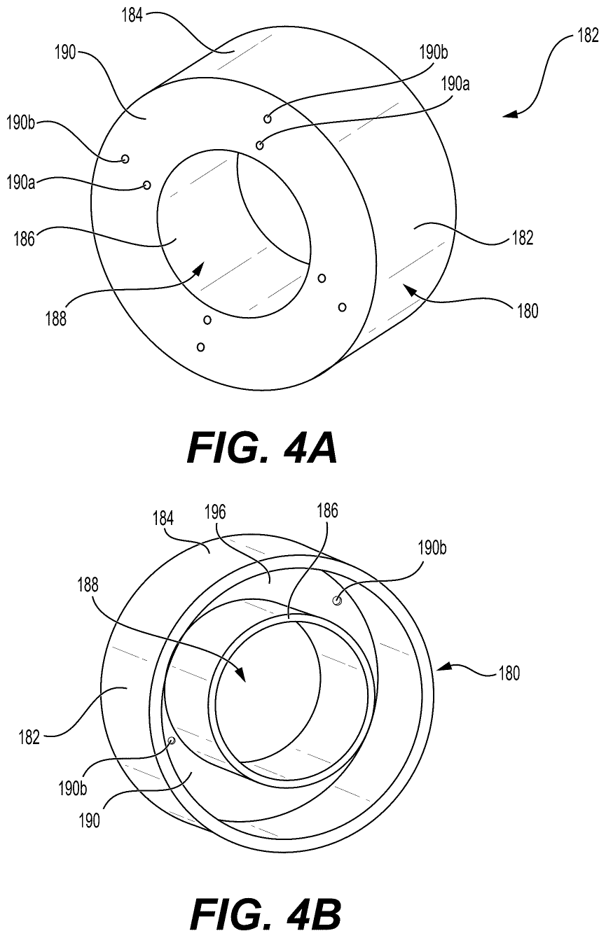

FIGS. 4A and 4B illustrate perspective views of second container 180. For example, FIG. 4A shows a perspective view of a distal end of second container 180 with distal cap 190 and inner and outer circumferences 184, 186, and FIG. 4B shows a perspective view of the proximal end of second container 180 with chamber 196. As shown in FIGS. 2, 4A-4B, 5A-5B, medical device 100 may include second container 180. Second container 180 may include a casing 182. Casing 182 may be an annular or O-shaped cylindrical structure defining an outer circumference 184 and an inner circumference 186. The open space within inner circumference 186 may define a central opening or passage 188. Passage 188 may be of any suitable shape and/or diameter that may receive distal portion 52a of shaft 52 of endoscope 50, so that shaft 52 may securely/snugly fit or otherwise be received within passage 188. In some embodiments, a surface of casing 182 surrounding passage 188 may be lined with, coated with, or otherwise include one or more frictious or adhesive materials to help secure the coupling between casing 182 and shaft 52 of endoscope 50.

As at least seen in FIGS. 2, 4A, 4B, 5A, and 5B, second container 180 may include a distal cap 190. Distal cap 190 may be O-shaped. Passage 188 may be extend through distal cap 190, so that distal cap 190 includes a central opening. Distal cap 190 may be positioned at distal ends of outer circumference 184 and inner circumference 186. Distal cap 190 may be integrally formed with casing 182 (e.g., fixedly attached to distal ends of outer circumference 184 and inner circumference 186). Distal cap 190 may seal a distal end of casing 182 and a proximal end of casing 152. Second container 180 may include a proximal cap 192, as shown in at least FIG. 2. Proximal cap 192 may include an aperture 194 at a center of the proximal cap 192. Proximal cap 192 may be O-shaped. Proximal cap 192 may seal a proximal end of casing 182. Aperture 194 may include a diameter approximately equal to a diameter of inner circumference 186. Aperture 194 may be sized and shaped to receive shaft 52 of endoscope 50. Second container 180 may include a chamber 196. Chamber 196 may be defined by a space between the inner circumference 186 and outer circumference 184 proximal to distal cap 190 and distal to proximal cap 192.

FIGS. 5A and FIG. 5B illustrate perspective views of medical device 100. For example, FIG. 5A illustrates a view of medical device 100 when viewed in a distal to proximal direction, and FIG. 5B illustrates a view of medical device 100 when viewed in a proximal to distal direction. FIG. 5A further shows balloon 174 and barrier layer 178 positioned within first chamber 170. As seen at least in FIGS. 5A-5B, a distal end of second container 180 may be coupled/affixed to a proximal end of first container 150. Additionally, FIG. 5A shows distal cap 160 with one-way valve(s) 160b, electrical strip 164, and light source(s) 164b. FIG. 5B further shows holes 190a of distal cap 190 and tubing 104 (e.g., tubes 104a, 104b, 104c, 104d) extending into balloon 174. Tubing 104 may be fluidly isolated from chamber 196. As seen in FIGS. 5A-5B, in some embodiments, first container 150 includes balloon 174. Balloon 174 may include a cylindrical and/or annular shape. Balloon 174 may be positioned within chamber 170. A radially exterior surface of balloon 174 may contact a radially interior surface of outer circumference 156. In some embodiments, at least a portion of the radially exterior surface of balloon 174 is fixed or otherwise adhered to the radially interior surface of outer circumference 156. While transitioning from an uninflated configuration to an inflated configuration, balloon 174 may expand radially inward toward, for example, inner circumference 154. Balloon 174 may extend from a proximal end of chamber 170 to a distal end of chamber 170 (e.g., from distal cap 160 to distal cap 190) and may be fixed to the proximal end and the distal end of chamber 170, which may help to prevent displacement. Balloon 174 may be at least partially inflated by supplying balloon 174 with a gas or fluid, for example, via syringe 102.

Chamber 170 may include a fluid cavity 176 positioned between a radially interior surface of balloon 174 and inner circumference 154. Fluid cavity 176 may be filled with a treatment agent, for example, a highly viscous fluid. Fluid cavity 176 may be configured to receive the highly viscous fluid (e.g., a first treatment agent or fluid) from second container 180.

Chamber 170 may include a barrier layer 178 positioned between balloon 174 and fluid cavity 176. Barrier layer 178 include a cylindrical and/or annular shape. Barrier layer 178 may extend from the proximal end of chamber 170 to the distal end of chamber 170. Barrier layer 178 may include one or more bellows 178a along a circumference of barrier layer 178. One or more bellows 178a may help barrier layer 178 collapse or otherwise move radially inward when the interior surface of balloon 174 urges barrier layer 178 radially inward while balloon 174 is transitioning from the deflated configuration to the inflated configuration. Due to pneumatic pressure, one or more bellows 178a of barrier layer 178 may help facilitate a greater force being applied to the treatment fluid relative to another barrier layer 178 without one or more bellows 178a. Barrier layer 178 may be configured to move radially inwards while balloon 174 is transitioning from the deflated state to the inflated state. As barrier layer 178 moves radially inward and balloon 174 expands radially inward, the pressure of the fluid (the first treatment agent or fluid) within fluid cavity 176 may increase.

Additionally, in some embodiments, distal cap 160 includes one or more through holes 160a. One or more through holes 160a may be fluidly connected with fluid cavity 176. Each of one or more through holes 160a may include or be coupled to a nozzle or a one-way valve 160b at a distal opening of each of one or more through holes 160a. Valve(s) 160b may be configured to prevent the first treatment agent or fluid within fluid cavity 176 from exiting through valve(s) 160b when the first treatment agent or fluid is at a first pressure (e.g., when balloon 174 is not exerting a force on the first treatment agent or fluid of fluid cavity 176). Valve(s) 160b may be configured to allow the first treatment agent or fluid within fluid cavity 176 to exit through valve(s) 160b when the first treatment agent or fluid is pressurized above the first pressure (e.g., when balloon 174 is exerting a force on the first treatment agent or fluid of fluid cavity 176). The first treatment agent or fluid within fluid cavity 176 may exit through valve(s) 160b when a sufficient amount of force is applied to the first treatment agent or fluid of fluid cavity 176 by balloon 174, such that the first treatment agent or fluid may be forced through valve(s) 160b (e.g., by the transition of balloon 174 from the deflated configuration to the inflated configuration). Medical device 100 may be positioned (e.g., via movement or deflection of shaft 52) within a bodily lumen or orifice so that the first treatment agent or fluid exiting through valve(s) 160b may be delivered to a treatment site (not shown).

Distal cap 190 may include one or more inner holes 190a. One or more inner holes 190a may be in fluid communication with fluid cavity 176 and chamber 196. Chamber 196 may be filled with the same first treatment agent or fluids fluid cavity 176, for example, additional first treatment agent or fluid. When balloon 174 is fully inflated, all or substantially all of the first treatment agent or fluid of fluid cavity 176 may be forced distally through valve(s) 160b. Transitioning balloon 174 from the inflated configuration to a deflated configuration may create a partial vacuum and/or negative pressure within fluid cavity 176. When fluid cavity 176 is negatively pressurized, the first treatment agent or fluid of chamber 196 may be pulled or drawn into fluid cavity 176 to at least partially refill fluid cavity 176 with first treatment agent or fluid. In these aspects, balloon 174 may be re-inflated to deliver more of the first treatment agent or fluid onto the treatment site. One or more inner holes 190a may each include a one-way valve (not shown) to help prevent the first treatment agent or fluid from flowing from fluid cavity 176 into chamber 196. Alternatively, chamber 196 may enclose a treatment agent or fluid (e.g., a second treatment agent or fluid) that is different from the first treatment agent or fluid.

Syringe 102 may be coupled to tubing 104. Tubing 104 may include one or more of tubes, for example, four tubes 104a, 104b, 104c, and 104d. Tubing 104 may branch out into one or more of tubes 104a, 104b, 104c, and 104d at a predetermined distance from the distal end of syringe 102. Although tubing 104 is described as including four tubes 104a, 104b, 104c, and 104d, tubing 104 may include any number of tubes, for example, one, two, three, five, or more tubes. Tubing 104 may extend from a distal end of syringe 102. Tubing 104 may be configured to deliver a gas or fluid (such as air) from syringe 102 to balloon 174. Tubing 104 may also be configured to remove a gas or fluid from balloon, and the removed gas or fluid may be received within syringe 102. The gas or fluid may be removed or delivered by manipulating syringe 102 (e.g., by pulling plunger 102a or compressing plunger 102a). Tubing 104 may extend along an exterior of shaft 52 of endoscope 50 from a proximal end of shaft 52 to a distal end of shaft 52.

Distal cap 190 may include one or more outer holes 190b and proximal cap 192 may include one or more holes 192a. There may be a 1:1:1 ratio of outer hole(s) 190b to hole(s) 192a to tube(s) 104a, 104b, 104c, and 104d. For example, if tubing 104 includes four tubes 104a, 104b, 104c, and 104d, then distal cap 190 may include four outer holes 190b. Similarly, proximal cap 192 may include four holes 192a. Holes 190b, 192a may be positioned radially outward relative to hole(s) 190a. Holes 190b, 192a may each be sized and shaped to receive a portion of one of tube(s) 104a, 104b, 104c, 104d. Each of tube(s) 104a, 104b, 104c, and 104d may extend through one of holes 192a, then extend across chamber 196, and then extend through one of holes 190b. A distal opening and/or distal end of each of tube(s) 104a, 104b, 104c, and 104d may be positioned within balloon 174.

Syringe 102 may include plunger 102a positioned at a proximal end of syringe 102. Tubing 104 may be in communication with balloon 174, so that when depressing plunger 102a of syringe 102, gas/fluid may flow in the proximal to distal direction through tube(s) 104 to transition balloon 174 into the inflated configuration. Additionally, tube(s) 104 may be in communication with balloon 174, so that when decompressing (e.g. retracting) plunger 102a of syringe 102, gas/fluid may flow in the distal to proximal direction through tubing 104 to transition balloon 174 into the deflated configuration.

During a medical procedure, medical device 100 may be utilized to deliver the first treatment agent or fluid to the treatment site. The first treatment agent or fluid may be may be a highly viscous fluid and may be an adhesive. First treatment agent or fluid may include, for example, fibrin, however the first treatment agent or fluid is not limited to including fibrin and may include any one fluid or combination of fluids used within medical procedures to be delivered to treatment sites. The first treatment agent or fluid may be curable when exposed to electromagnetic radiation, such as ultraviolet (UV) radiation or visible light (VIS) radiation. The treatment agent or fluid may include one or more biomaterials, for example, including one or more photo initiators.

During a medical procedure, a distal end of endoscope 50 may be positioned near and/or adjacent to the treatment site so that the first treatment agent or fluid exiting valves 160b may be deposited onto the treatment site. The first treatment agent or fluid may be deposited onto the treatment site by compressing syringe 102, so that air flows through tubing 104 in the proximal to distal direction to transition balloon 174 into the inflated configuration. As balloon 174 is transitioned into the inflated configuration, the first treatment agent or fluid may be forced through valves 160b to be delivered to the treatment site. After the first treatment agent or fluid has been delivered to the treatment site, UV/VIS radiation may be emitted from one or more light sources 164b of electrical strip 164 to help cure the first treatment agent or fluid. For example, one or more light sources 164b of electrical strip 164 may be 4 W lamps emitting UV radiation at a 365 nm wavelength over a 120 cm² area of the treatment site. Alternatively or additionally, one or more light sources 164b of electrical strip 164 may be 150 W halogen lamps for emitting visible light over an 8πcm² area of the treatment site. The visible light may be concentrated via a gooseneck to increase effectiveness. The first treatment agent or fluid may be cured via one or more light sources of electrical strip 164 within about ten seconds (+/−ten seconds). Alternatively or in addition to light source(s) 164b of electrical strip 164, the first treatment agent or fluid may be cured by light source(s) 56 positioned at a distal face of endoscope 50. The first treatment agent or fluid may also be cured by other mechanisms such as, but not limited to, pH of the anatomy, moisture absorption, and time.

After the first treatment agent or fluid has been delivered to the treatment site, syringe 102 may be decompressed (e.g., by retracting plunger 102a), which may retract or pull air from balloon 174 into syringe 102 and help to transition balloon 174 from the inflated configuration to the deflated configuration. As balloon 174 is transitioned from the inflated configuration to the deflated configuration, balloon 174 and barrier layer 178 may retreat or otherwise move radially outward toward outer circumference 156. As balloon 174 and barrier layer 178 retreat radially outward, negative pressure may be created within fluid cavity 176 and a treatment agent or fluid from chamber 196 (e.g., an additional amount of the first treatment agent or fluid or a different treatment agent or fluid) may be pulled into fluid cavity 176 to refill fluid cavity 176, for example, for additional treatment of the treatment site.

Additional treatment agent or fluid may be delivered to the treatment site by repeating the abovementioned steps. For example, these steps may be repeated until the treatment site no longer requires additional treatment agent or fluid (e.g., when a protective layer of cured treatment agent or fluid has been applied to the treatment site to help prevent delayed bleeding, potential perforations, and/or stricture formation). Alternatively, these steps may be repeated until the treatment agent(s) or fluid(s) within chamber 196 and fluid cavity 176 has been fully depleted.

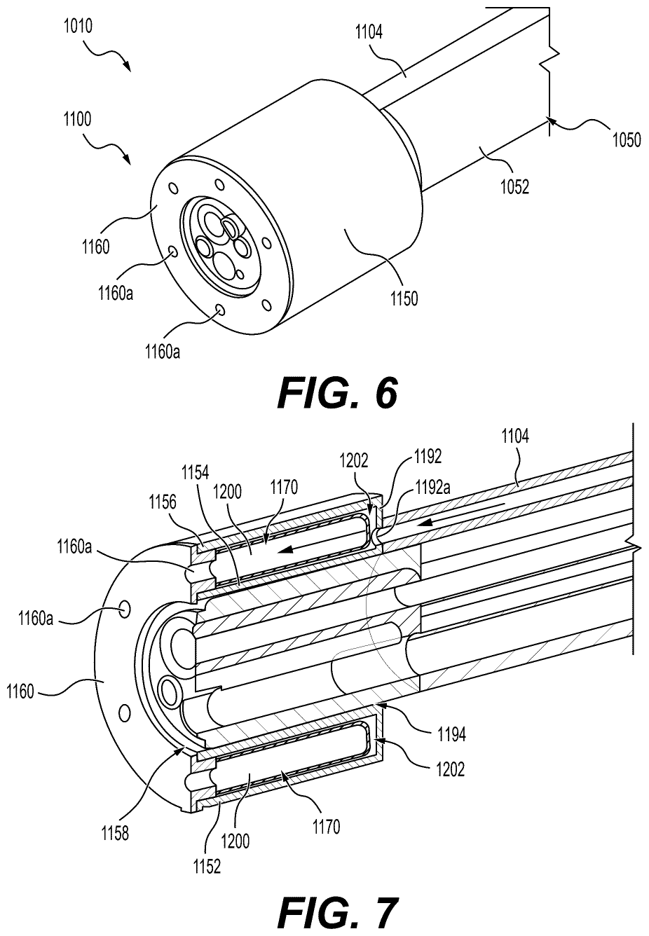

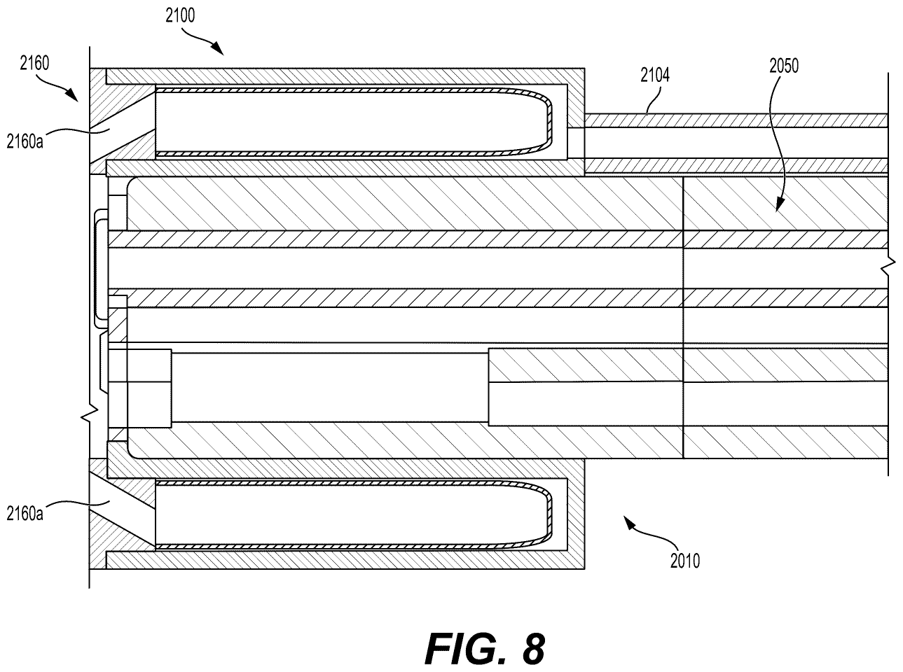

FIGS. 6 and 7 illustrate perspective views of a medical device 1100. FIG. 6 shows a medical device 1100 coupled/affixed to a distal end of an endoscope 1050 and further illustrates a first container 1150 and a distal cap 1160 including hole(s) 1160a positioned at a distal end of first container 1150. FIG. 7 shows a cross sectional view of medical device 1100 and further illustrates an inner circumference 1154, an outer circumference 1156, and tubing 1104 in communication with a chamber 1170 with pouch(es) 1200 disposed within chamber 1170. As shown in FIG. 6, one or more portions of medical device 1100 may be coupled to another or second medical device, for example, endoscope 1050, to form a medical system 1010. Elements of medical system 1010 and medical device 1100 are not necessarily shown to scale and certain components of medical system 1010 may be omitted from the figures for clarity purpose. Endoscope 1050 may include a generally cylindrical tubular shaft 1052. Endoscope 1050 and shaft 1052 may have any feature of endoscope 50 and shaft 52, respectively. Although not shown, medical system 1010 may include a syringe (such as syringe 102) to perform some or all of the functions of syringe 102. The syringe may be coupled to tubing 1104. Tubing 1104 may include one or more tubes (such as tubes 104a, 104b, 104c, and 104d). Tubing 1104 may include any of the features of tubing 104.

As shown in FIGS. 6 and 7, medical device 1100 may include a first container 1150. First container 1150 may include a casing 1152. Casing 1152 may be an annular or O-shaped cylindrical structure defining an inner circumference 1154 and an outer circumference 1156. The open space within inner circumference 1154 may define a central opening or passage 1158. Passage 1158 may be of any suitable shape and/or diameter that may receive a distal portion of shaft 1052 of endoscope 1050, so that shaft 1052 may securely/snugly fit within passage 1158. In some embodiments, a surface of casing 1152 surrounding passage 1158 may be lined or coated with (or otherwise include) frictious or adhesive material to further secure the coupling between casing 1152 and shaft 1052 of endoscope 1050.

First container 1150 may include a distal cap 1160. Passage 1158 may be defined through distal cap 1160, so that distal cap 1160 includes a donut-like shape. Distal cap may be O-shaped. Distal cap 1160 may be positioned at a distal end of inner circumference 1154 and at a distal end of outer circumference 1156. Distal cap 1160 may be integrally formed with casing 1152 (e.g., fixedly attached the distal ends of inner circumference 1154 and outer circumference 1156) and may seal a distal end of casing 1152. First container 1150 may include chamber 1170. Chamber 1170 may be defined by a space between the inner circumference 1154 and outer circumference 1156 proximal to distal cap 160.

First container 1150 may include a proximal cap 1192. Proximal cap 1192 may include an aperture 1194 at a center of the proximal cap 1192. Proximal cap 1192 may be O-shaped. Aperture 1194 may include a diameter approximately equal to a diameter of inner circumference 1154. Aperture 1194 may be sized and shaped to receive shaft 1052 of endoscope 1050. Proximal cap 1192 may be positioned at a proximal end of chamber 1170, and may be positioned at a proximal end of inner circumference 1154 and a proximal end of outer circumference 1156. Proximal cap 1192 may be fixedly attached or releasably coupleable at the proximal ends of inner circumference 1154 and outer circumference 1156. Proximal cap 1192 may seal a proximal end of casing 1152. Proximal cap 1192 may include one or more chamber holes 1192a. Chamber hole(s) 1192a may extend from a proximal facing surface of proximal cap 1192 to a distal facing surface of proximal cap 1192. Chamber holes 1192a may be in communication with chamber 1170. Distal ends of tubing 1104 may be fixedly attached or releasably attached to proximal ends of chamber hole(s) 1192a so that tubing 1104, chamber hole(s) 1192a, and chamber 1170 are in communication.

Chamber 1170 may include one or more pouches 1200. Pouch(es) 1200 may be configured to store a fluid, for example, a treatment agent or fluid. The treatment agent or fluid may be same as and/or have any of the features of the first treatment agent or fluid. The treatment agent or fluid may include one or more biomaterials, for example, including one or more photo initiators. Pouch(es) 1200 may be fixedly attached to a proximal-facing surface of distal cap 1160 and/or fixedly attached to a distal end of chamber 1170. A portion of chamber 1170 not occupied by pouch(es) 1200 may define a free-space 1202. Increasing a pressure of free-space 1202 may apply force to pouch(es) 1200. Distal cap 1160 may include one or more chamber holes 1160a. Chamber hole(s) 1160a may extend from a proximal facing surface of distal cap 1160 to a distal facing surface of distal cap 1160. Each of chamber holes 1160a may be in fluid communication with a volume of pouch(es) 1200 so that the treatment agent or fluid contained within pouch(es) 1200 may flow in the proximal to distal direction through chamber holes 1160a when a force is applied to pouch(es) 1200. Chamber holes 1160a may parallel with a central longitudinal axis of endoscope 1050 or shaft 1052.

When using medical system 1010 during a medical procedure, medical device 1100 and a distal end of endoscope 1050 be positioned near or adjacent to a treatment site. Medical device 1100 may be positioned close enough to the treatment site so that the treatment agent or fluid exiting through the distal end of chamber hole(s) 1160a is deposited onto the treatment site. The treatment agent or fluid may be ejected from medical device 1100 by compressing the syringe (e.g., syringe 102) of medical system 1010. Compressing the syringe may deliver a gas (such as air) or a fluid through tubing 1104 in a proximal to distal direction to increase a pressure of chamber 1170 and/or increase a pressure of free-space 1202. Increasing the pressure of chamber 1170 and/or free-space 1202 may apply a force against pouch(es) 1200. At a predetermined pressure within chamber 1170 and/or free-space 1202, pouch(es) 1200 may be compressed, forcing the treatment agent or fluid through chamber hole(s) 1160a onto the treatment site. Although not shown, medical device 1100 may include one or more light sources, for example, to help cure the delivered treatment agent or fluid. The treatment agent or fluid may also be cured by other mechanisms such as, but not limited to, pH of the anatomy, moisture absorption, and time. Additionally, after delivering a portion of the treatment agent in pouch(es) 1200, medical system 1010 may be repositioned, for example, by repositioning endoscope 1050, and additional treatment agent may be delivered, for example, by delivering additional gas/fluid to free-space 1202.

FIG. 8 illustrates a cross-section view of medical system 2010. FIG. 8 shows a distal cap 2160 of a medical device 2100 and further shows the angled chamber holes 2160a of distal cap 2160. Medical system 2010 may include any of the features of medical system 1010, except as noted below. For example, medical system 2010 may include a medical device 2100 and an insertion device 2050 (e.g., an endoscope). Medical device 2100 may include any features of medical device 1100. Components and elements of medical system 2010 analogous or identical to components and elements of medical system 1010 share the same element number but with an additional 1000 added to the element number of medical system 1010 (for example, tubing 1104 of medical system 1010 would be tubing 2104 of medical system 2010). Medical device 2100 differs from medical device 1100 in that chamber holes 2160a, from a proximal facing surface of distal cap 2160 to a distal facing surface of distal cap 2160, extend distally and radially inward toward a central longitudinal axis of endoscope 2050. Angling chamber holes 2160a radially inward as holes 2160a extend in a proximal to distal direction may help the operator in depositing more of first treatment agent or fluid in a smaller area compared to the chamber holes 1160a of medical system 1010. For example, with chamber holes 2160a being angled radially inward (e.g., toward a central longitudinal axis of medical device 2100 and/or a central longitudinal axis of insertion device 2050), the treatment agent may be delivered closer to the central longitudinal axis of medical system 2010. In other words, angling chamber holes 2160a may increase the accuracy and/or precision of the deposition of the treatment agent or fluid onto the treatment site.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a container, the container including a chamber and a distal cap;
wherein, the distal cap of the container includes one or more holes therethrough
wherein the chamber includes a balloon or a pouch, and a fluid cavity, wherein the balloon or pouch radially surrounds the fluid cavity,
wherein transitioning the balloon or pouch from a deflated configuration to an inflated configuration or from the inflated configuration to the deflated configuration urges a fluid within the fluid cavity through the one or more holes of the distal cap.

2. The medical device of claim 1, wherein the chamber includes a balloon, wherein the medical device further comprises a barrier layer positioned between the balloon and the fluid cavity.

3. The medical device of claim 2, wherein the barrier layer includes one or more bellows.

4. The medical device of claim 1, wherein the chamber includes a balloon and a barrier layer positioned between the balloon and the fluid cavity, wherein the container is a first container, wherein the chamber is a first chamber, wherein the medical device further includes a second container, wherein the second container includes a second chamber, and wherein the second container includes one or more holes in fluid communication with the fluid cavity of the first chamber.

5. The medical device of claim 4, wherein a transition of the balloon from the inflated configuration to the deflated configuration urges another fluid from the second chamber into the fluid cavity of the first chamber.

6. The medical device of claim 4, wherein the fluid cavity includes the fluid, wherein the fluid is fibrin, wherein the second container includes the second chamber, wherein the second chamber includes the fluid.

7. The medical device of claim 4, wherein the first container includes a first outer circumference and a first inner circumference, wherein the balloon, the barrier layer, and the fluid cavity are positioned between the first outer circumference and the first inner circumference.

8. The medical device of claim 7, wherein the medical device is positioned at a distal end of a shaft of an endoscope, wherein the first inner circumference includes a first open space radially inward of the first inner circumference, and wherein the first open space defines a first passage sized and shaped to receive the shaft of the endoscope.

9. The medical device of claim 8, wherein the second container includes a second outer circumference and a second inner circumference, wherein the second inner circumference includes a second open space radially inward of the second inner circumference, and wherein the second open space defines a second passage sized and shaped to receive the shaft of the endoscope.

10. The medical device of claim 7, wherein the distal cap of the first container includes a circular electrical strip with one or more light sources thereon, and wherein the circular electrical strip is positioned radially outward of the first inner circumference and radially inward of the first outer circumference.

11. The medical device of claim 10, wherein the first container includes a channel positioned on an exterior surface of the first container, wherein the channel includes a lumen that includes an electrical conduit, wherein the circular electrical strip includes a tab extending radially outward from the electrical strip, and wherein the electrical conduit is electrically connected to the tab of the electrical strip to supply electrical power to the one or more light sources of the electrical strip.

12. The medical device of claim 9, wherein the second container includes a distal cap and a proximal cap, the distal cap includes one or more distal outer holes and one or more distal inner holes and the proximal cap includes one or more proximal holes.

13. The medical device of claim 12, wherein the medical device includes one or more tubes configured to provide and/or remove gas or fluid from the balloon, wherein a distal opening of each of the one or more tubes is in fluid communication with the balloon, and wherein the one or more distal outer holes and the one or more proximal holes are each sized and shaped to receive a portion of one of the one or more tubes.

14. The medical device of claim 12, wherein the distal cap of the first container is positioned near distal ends of the first inner circumference and the first outer circumference, wherein the distal cap of the second container is positioned at proximal ends of the first inner circumference and the first outer circumference to seal the first container, wherein the distal cap of the second container is positioned at distal ends of the second inner circumference and the second outer circumference, wherein the proximal cap is positioned at proximal ends of the second inner circumference and the second outer circumference to seal the second container, and wherein a proximal end of the first container is affixed to a distal end of the second container.

15. The medical device of claim 1, the one or more holes of the distal cap extend, in a proximal direction to a distal direction, radially inward relative to a central longitudinal axis of the medical device.

16. A medical system comprising:
a first container, wherein the first container includes a first chamber and a distal cap;
a second container, wherein the second container includes a second chamber, a distal cap, and a proximal cap;
a syringe; and
one or more tubes extending from a distal end of the syringe to the first container,
wherein the first chamber includes a balloon and a fluid cavity, wherein the balloon radially surrounds the fluid cavity,
wherein the syringe, the one or more tubes, and the balloon are in fluid communication,
wherein transitioning the balloon from a deflated configuration to an inflated configuration by delivering a fluid or a gas from the syringe urges fluid within the fluid cavity through one or more holes of the distal cap.

17. The medical system of claim 16, wherein the one or more tubes extend through the distal cap and the proximal cap of the second container.

18. The medical system of claim 17, wherein the one or more tubes are fluidly isolated from the second chamber.

19. The medical system of claim 18, wherein the distal cap of the second container includes one or more holes, wherein the fluid cavity, the one or more holes of the distal cap of the second container, and the second chamber are in fluid communication.

20. A medical system comprising:
a first container, wherein the first container includes a first chamber and a distal cap;
a second container, wherein the second container includes a second chamber;
a syringe; and
one or more tubes extending from a distal end of the syringe,
wherein the first chamber includes a balloon and a fluid cavity, wherein the balloon radially surrounds the fluid cavity, wherein the first chamber includes a barrier layer positioned between the balloon and the fluid cavity,
wherein a distal end of the second container is affixed to a proximal end of the first container,
wherein the syringe, the one or more tubes, and the balloon are in fluid communication,
wherein transitioning the balloon from a deflated configuration to an inflated configuration by manipulating the syringe urges fluid within the fluid cavity through one or more holes of the distal cap.
